(19) United States
(12) Patent Application Publication
(10) Pub. No.: US 2023/0149449 A1
(43) Pub. Date: May 18, 2023

(54) ION CAPTURE COMPOSITION FOR INGESTION

(71) Applicant: NO-DIUM LLC, LAKE CHARLES, IA (US)

(72) Inventors: Dyllan Christopher Muller, Little Elm, TX (US); Austin Reid Wolf, Lake Charles, LA (US)

(21) Appl. No.: 17/919,468

(22) PCT Filed: Apr. 16, 2021
(86) PCT No.: PCT/US2021/027741

§ 371 Date: Oct. 17, 2022

Related U.S. Application Data
(60) Provisional application No. 63/010,850, filed on Apr. 16, 2020.

(51) Int. Cl. A61K 33/24; A61K 9/00; A61K 9/10; A61P 39/04; B01J 20/02; B01J 39/02; B01J 39/12

(52) U.S. Cl. CPC A61K 33/24; A61K 9/0056; A61K 9/0095; A61K 9/10; A61P 39/04; B01J 20/0211; B01J 39/02; B01J 39/12

(57) ABSTRACT

The invention is directed to ion-capture compositions for ingestion and methods of use thereof. An aspect of the invention is drawn towards a method for selectively capturing at least one ion from the digestive system of a subject, the method comprising administering orally to the subject the ion-capture composition.

This application claims the benefit of and priority to U.S. Patent Application No. 63/010,850 filed on Apr. 16, 2020, the contents of which is herein incorporated by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to ion-capture compositions for ingestion and methods of use thereof.

### BACKGROUND OF THE INVENTION

High sodium intake has been correlated with a higher risk of heart disease as well as other associated health problems, such as hypertension. Heart disease has consistently been the leading cause of death around the world for the past 20 years, as well as a major cause of disability. (World Health Organization. “WHO Reveals Leading Causes of Death and Disability Worldwide: 2000-2019.” Dec. 9, 2020. Currently, 16% of total global deaths from all causes are related to heart disease, with an increasing trend observed in many regions. Buying low sodium foods can be prohibitively expensive for many individuals. Furthermore, low sodium options do not provide as much variation as their high sodium counterparts.

### SUMMARY OF THE INVENTION

An aspect of this invention is drawn towards an ion-capture composition. In one embodiment, the composition comprises an ion-capture compound, wherein the compound traps at least one ion from a sample ingested by a subject. In an embodiment, the ion-capture composition comprises the formula M′M″ₓA₂B₃O₁₂, wherein the compound is in NZP form. In a further embodiment, the ion-capture composition comprises the formula M′M″ₓA₂B₃O₁₂, wherein M′ comprises a lithium ion, sodium ion, potassium ion, hydrogen ion, hydronium ion, rubidium ion, cesium ion, magnesium ion, calcium ion, strontium ion, barium ion, or a combination thereof; M″ comprises a sodium ion, potassium ion, hydrogen ion, hydronium ion, or a combination thereof; A comprises tin, germanium, titanium, zirconium, hafnium, aluminum, chromium, nobium, tantalum, scandium, or a combination thereof; B comprises phosphorous, silicon, aluminium, sulfur, or mixtures thereof; and “x” has a value of 0 to about 3. In a further embodiment, the composition comprises hydrogen zirconium phosphate, or HZr₂(PO₄)₃.

In an embodiment, the composition further comprises a pharmaceutically acceptable carrier. In an embodiment, the ion-capture compound comprises microscopic cubic crystals. In an embodiment, the ion-capture composition comprises a powder, a granule, a crystal, a suspension, an emulsion, a solution, or a resin. In a further embodiment, the ion-capture composition is provided as a powder, a tablet, a capsule, or a pre-made suspension. In a further embodiment, the composition in powder form is combined with a solution, thereby forming a suspension. In another embodiment, the composition in suspension form comprises a buffer.

In an embodiment the composition is formulated for oral administration. In another embodiment, the composition is formulated to be ingested by the subject.

In an embodiment, the ion-capture composition comprises a compound that traps at least one ion from a sample ingested by a subject, wherein the at least one ion comprises a monovalent ion. In an embodiment, the at least one ion comprises a sodium ion. In another embodiment, the at least one ion comprises a sodium ion, a lithium ion, a rubidium ion, or a cesium ion.

In an embodiment, the ion-capture composition comprises a compound that traps at least one ion from a sample ingested by a subject, wherein the ion-capture comprises chemical capture, physical capture, or both. In an embodiment, the compound captures an ion through ion-exchange, trapping ions within the crystal structure of the substance, adsorption, or chelation.

In another embodiment, the ion-capture composition is a pharmaceutical composition. In a further embodiment, the composition comprises a buffer.

An aspect of the invention is drawn towards a method for capturing at least one ion from a sample ingested by a subject. In an embodiment, the method comprises administering orally to the subject the ion-capture composition. For example, the composition is administered before, concurrently with, or after the subject ingests a sample comprising one or more ions. In a further embodiment, the at least one ion comprises a monovalent ion. In another embodiment, the at least one ion comprises a sodium ion. In another embodiment, the at least one ion comprises a sodium ion, a lithium ion, a rubidium ion, or a cesium ion. In another embodiment, the method of capturing at least one ion comprises chemical capture, physical capture, or both. In another embodiment, the compound captures an ion through ion-exchange, trapping ions within the crystal structure of the compound, adsorption, or chelation.

An aspect of the invention is drawn towards a method for selectively capturing at least one ion from the digestive system of a subject, the method comprising administering orally to the subject the ion-capture composition. In an embodiment, the method includes where least one ion is captured in the digestive tract. In another embodiment, the subject is administered about 1 milligram to about 100 grams per day.

### BRIEF DESCRIPTION OF THE FIGURES

FIG. 1 shows a graph of sodium concentration over time for 1 g Zirconium Phosphate: 45.7 mg sodium test.

FIG. 2 shows a graph of sodium concentration over time for 2 g Zirconium Phosphate: 45.7 mg sodium test.

FIG. 3 shows a graph of sodium concentration over time for 4 g Zirconium Phosphate: 45.7 mg sodium test.

FIG. 4 shows a graph of sodium concentration over time for 4 g Zirconium Phosphate: 45.7 mg sodium broth test.

FIG. 5 shows an PXRD spectrum.

FIG. 6 is an electron micrograph of an SEM image of the hydrogen zirconium phosphate synthesized; the scale bar is 500 nm.

FIG. 7 is an electron micrograph of an SEM image of the hydrogen zirconium phosphate synthesized; the scale bar is 2 nm.

FIG. 8 is an electron micrograph of an SEM image of the hydrogen zirconium phosphate synthesized; the scale bar is 2 nm.

FIG. 9 is an electron micrograph of an SEM image of the hydrogen zirconium phosphate synthesized; the scale bar is 3 nm.

FIG. 10 is an electron micrograph of an SEM image of the hydrogen zirconium phosphate synthesized; the scale bar is 500 nm.

FIG. 11 is an electron micrograph of an SEM image of the hydrogen zirconium phosphate synthesized; the scale bar is 2 μm.

FIG. 12 is an electron micrograph of an SEM image of the hydrogen zirconium phosphate synthesized; the scale bar is 10 μm.

FIG. 13 is an electron micrograph of an SEM image of the hydrogen zirconium phosphate synthesized; the scale bar is 500 nm.

FIG. 14 is an electron micrograph of an SEM image of the hydrogen zirconium phosphate synthesized; the scale bar is 400 nm.

FIG. 15 is an energy-dispersive x-ray spectroscopy elemental analysis of the hydrogen zirconium phosphate synthesized; showing a Zr/P ratio of about 2:3.

### DETAILED DESCRIPTION OF THE INVENTION

The invention is directed to compositions and methods of selectively removing ions from a sample ingested by a subject comprising an ion-capture compound that traps at least one ion from a sample ingested by a subject. For example, the ion-capturing composition orally administered to a subject before, concurrently, or after the subject ingests an ion-containing sample.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Ion-Capture Composition for Ingestion

Aspects of the invention are directed towards an ion-capturing composition for ingestion. As used herein, a composition can refer to any mixture. It can be a solution, suspension, liquid, powder, granule, crystal, emulsion, or a resin.

In embodiments, an ion-capturing composition can comprise a compound that traps at least one ion from a sample ingested by a subject. A “subject” can refer to any higher organism that is susceptible to a disease or condition. Examples of such higher organisms can include, without limitation, mice, rats, rabbits, dogs, cats, horses, cows, pigs, sheep, fish and reptiles. In some embodiments, “subject” can refer to a human being.

An “ion-capture composition” also can be referred to as an “ion-removing composition”. In some embodiments, the ion-capture composition can be a compound, or mixture of compounds, that can capture or bind an ion (such as, by physical, electromagnetic, chemical, electromagnetic, or other means, or a combination thereof), thus allowing the ion to be removed from a sample ingested by a subject. This “ion removing composition” can consist of both compounds that perform the actual capturing or binding (such as hydrogen zirconium phosphate), and substances that contribute to the form, size, and shape of the “ion-capture composition” as a whole (such as a silica bead, gels, and the like). The “ion removing composition” can comprise the compounds that perform the actual capturing or binding (such as hydrogen zirconium phosphate) and, optionally, a pharmaceutically acceptable carrier.

As used herein, a “substance” can refer to any type of drug, biologically active agent, biological substance, chemical substance, or biochemical substance that can be administered in a therapeutically effective amount to a subject. For example, the ion-capturing composition comprises a compound of formula M′M″ₓA₂B₃O₁₂ in NZP (sodium zirconium phosphate) form, wherein M′ is the ion to be captured by the compound. such as a lithium ion, sodium ion, potassium ion, hydrogen ion, hydronium ion, rubidium ion, cesium ion, magnesium ion, calcium ion, strontium ion, barium ion, or mixtures thereof; M″ is a sodium ion, potassium ion, hydrogen ion, hydronium ion, or mixtures thereof; A is tin, germanium, titanium, zirconium, hafnium, aluminum, chromium, nobium, tantalum, scandium, or mixtures thereof; B is phosphorous, silicon, aluminum, sulfur, or mixtures thereof; “x” has a value of 0 to about 3. See, for example, Hawkins, H. T., et al. “Synthesis and characterization of uranium (IV)-bearing members of the [NZP] structural family.” Chemistry of materials 11.10 (1999): 2851-2857 and Alamo, James, and Rustum Roy. “Crystal chemistry of the NaZr₂(PO₄)₃, NZP or CTP, structure family.” Journal of materials science 21.2 (1986): 444-450, each of which are incorporated by reference herein in their entireties.

As another example the ion-capture composition comprises the compound or compounds according to FIG. 15. For example, FIG. 15 refers to an energy-dispersive x-ray spectroscopy elemental analysis of the hydrogen zirconium phosphate synthesized, and shows a Zr/P ratio of about 2:3.

Zirconium Phosphate, HZr₂(PO₄)₃ is not the only compound that can selectively remove sodium ions. Without wishing to be bound by theory, there are other zirconium compounds that can perform similarly. In embodiments, the pores in the ion exchanging crystals can act as a molecular sieve, allowing the passage of, for example, sodium overwhelmingly more than any other ions. In embodiments, this can entail a pore diameter of around 2.1 to 2.7 Angstroms, for example. Other non-limiting examples of compounds comprise hydrogen zirconium phosphate (HZP), HZr₂(PO₄)₃, another zirconium phosphate, or similar compounds comprising the formula HA₂B₃O₁₂, wherein A can be tin, germanium, titanium, zirconium, hafnium, aluminum, chromium, nobium, tantalum, scandium, or mixtures thereof; and B comprises phosphorous, silicon, aluminum, sulfur, or mixtures thereof.

In embodiments, the ion-capture composition can comprise a pharmaceutically acceptable carrier. As used herein, a “pharmaceutically acceptable carrier” can refer to a substance that aids the administration of an active agent to and absorption by a subject. Pharmaceutically acceptable carriers can be included in compositions as described herein without causing a significant adverse toxicological effect on the subject.

In embodiments, the ion-capture composition can comprise one or more additional active ingredients. For example, an “active ingredient” can refer to any substance that has activity in a biological system or organism.

In embodiments, the ion-capture compound can comprise microscopic cubic crystals. For example, the term “crystal” can refer to a composition comprising an inorganic compound in crystalline form.

In embodiments, the ion-capture composition can further comprise a compound, such as zirconium phosphate, which allows for the entrapment and immobilization of the ions from an ingested sample. As used herein, the term “immobilization” can refer to the attachment or entrapment, either chemically or otherwise, of a material (such as ions from an ingested sample) to another entity (such as to the ion-capture composition) in a manner that restricts the movement of the material. The ion-capture composition can have a high-surface area such that contact with the ingested sample intended for ion removal is maximized. The ion-capture composition can be a powder, a granule, a crystal, a suspension, an emulsion, a solution, polymeric beads (such as polyacrylamide beads), a gel (such as agarose, carrageenan, or alginate) or a resin. “Solution” can refer to a mixture whose particles can be evenly distributed such that the relative concentrations of the components are the same throughout. “Powder” can refer to a composition that contains finely dispersed solid particles that are relatively free flowing and are not dissolved or suspended in a solvent. “Granule” can refer to a particle which contains a core, an active agent, and optionally at least one coating layer. For example, the granule can be spherical, cylindrical, disk-shaped. “Emulsion” can refer to a dispersed system containing at least two immiscible liquid phases (e.g., oil and water) that is stabilized with an emulsifying agent to form a single phase.

In embodiments, the ion-capture composition can comprise polymeric beads, which can have pores allowing the entry of gastrointestinal contents but preventing the exit of the ion capturing substance, such as zirconium phosphate. In other embodiments, the ion-capture composition can be silica sol, silica beads, a porous polymer, polymeric beads, or a gel. Such compositions are known in the art. See, for example, Oi, Takao, et al. “Synthesis of Zirconium Phosphate, HZr₂(PO₄)₃, in Pores of Silica Beads and Some Ion Exchange Separation Properties of the Composite Obtained.” Separation Science and Technology 44.15 (2009): 3679-3697, which is incorporated by reference herein in its entirety.

Aspects of the invention are further drawn to pharmaceutical compositions comprising an ion-capture composition described herein. As used herein, the term “pharmaceutical composition” can refer to a mixture or dispersion comprising the ion-capture composition to be administered to a subject, eg, a human, for the prevention, treatment or control of a particular disease or condition that the subject has.

For example, a pharmaceutical composition comprising an ion-capture composition can be administered to a subject in need thereof. As used herein, “administer,” “administering or “administration,” which can be used interchangeably, can refer to the delivery of a compound or of a pharmaceutical composition containing the compound to a subject in a manner suitable for the treatment of a particular disease or condition, such as heart disease or hypertension. “Administration” can be effected in one dose, continuously or intermittently throughout the course of treatment. Methods of determining the most effective means and dosage of administration are known to those of skill in the art and will vary with the composition used for therapy, the purpose of the therapy, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue.

In embodiments, the ion-capture composition can be administered orally. “Oral administration” can refer to a route of administration through the oral cavity or mouth. Thus, the ion-capture composition can be formulated to be ingested by the subject. “Ingested” can refer to the action of taking a substance into the body by swallowing. In embodiments, the ion-capture composition can be provided as a powder, a tablet, a capsule, or a pre-made suspension. A “tablet” can refer to an entity that comprises active substances and excipients, usually in powder form, pressed or compacted from a powder into a solid dose. A “capsule” can refer to a softgel, caplet, or any other encapsulated dosage form known to practitioners in the art. A “suspension” can refer to a heterogeneous mixture of a solid and liquid. Such compositions are known in the art. For example, see Keyser, et al., “MICROPOROUS ZIRCONIUM SILICATE FOR THE TREATMENT OF HYPERKALEMIA” U.S. application Ser. No. 14/036,489 which is incorporated by reference herein in its entirety.

In embodiments, the pharmaceutical composition comprises a suspension, wherein powder form is combined with a solution.

In embodiments, the ion-capture composition can capture one or more ions. An “ion” can refer to any atom, molecule, or other object that has a net electrical charge formed, for example, by addition or removing electrons from the object. For example, the ion comprises a sodium ion, lithium ion, potassium ion, hydrogen ion, hydronium ion, rubidium ion, cesium ion, magnesium ion, calcium ion, strontium ion, or barium ion.

The term “ion exchange” can refer to the reversible exchange of ions between one composition, such as a solid, and another, such as a fluid.

The ion can be a monovalent ion or a polyvalent ion. For example, the polyvalent ion can be a divalent ion.

In embodiments, the ion-capture composition can capture an ion through chemical means, physical means, or both. For example, a physical means can refer to physiosorption. For example, a chemical means can refer to chemisorption. In one embodiment, the physical means encompass ions experiencing an electronegative attraction towards the pores of the ion-capturing composition; once the composition has pulled the ions in, the small size of the pores act as a physical barrier, making it difficult for the ions to escape the electronegative pull of the pores.

In embodiments, the ion-capture composition can further comprise a buffering agent or a buffering solution. As used herein, a “buffer,” “buffering agent,” or “buffering solution” can refer to an agent or chemical that neutralizes either acids or bases thus stabilizing pH. Non-limiting examples of buffering agents include potassium citrate and calcium carbonate. As used herein, “potassium citrate” can refer to a compound with the formula C₆H₅K₃O₇. In embodiments, the buffering agent can be potassium citrate powder. “Calcium carbonate” can refer to a compound with the formula CaCO₃. For example, the buffering agent or buffering solution and the ion-capture compound can be mixed together into a tablet, capsule, or suspension. For example, the buffering agent or buffering solution can control the pH of the ion-rich sample ingested. For example, when hydrogen zirconium phosphate performs ion exchange on the ingested sample, hydrogen ions are released. Thus, a food-safe buffer can be needed. As one example, the buffer can be potassium citrate; however, the skilled artisan will recognize that other buffers can be utilized. See, for example, Title 21, Chapter 1, Subchapter B, part 184 of the Code of Federal Regulations.

In embodiments, the ion-capture composition can be provided as a dietary item. For example, the term “dietary item” can refer to any product that undergoes at least one processing or culinary step prior to distribution and is consumed by a subject. Non-limiting examples of processing and culinary steps include mixing, cooking, baking, heating, chopping, chilling, freezing, packaging, canning, bagging, and storing. Non-limiting examples of dietary items include food products, dietary ingredients, medical foods, functional foods, beverages, dietary supplements, vitamins, minerals, and combinations thereof.

In embodiments, the ion-capture composition can be used in food processing or in the production of a food product or drink product. As used herein, the term “food processing” can refer to all types of processing used for this, such as mixing, cooking, baking, heating, chopping, grinding, slicing, chilling, freezing, packaging, canning, bagging, and storing. “Food product” can refer to any substance, preparation, composition or object that is suitable for consumption, nutrition, oral hygiene or pleasure, and which is intended to be introduced into the human or animal oral cavity, to remain there for a certain period of time and then to either be swallowed or to be removed from the oral cavity again. These products include ail substances or products intended to be ingested by humans or animals in a processed or a semi-processed state. As used herein, the term “drink product” can refer to a composition in the form of an ingestible substance which, at the temperature it is properly stored and ingested, is in a free-flowing liquid form, and which will not be chewed prior to swallowing.

Method of Use

Aspects of the invention are also directed towards methods for using the ion-capture composition, such as to remove an ion from an ion-containing sample. For example, the ion-capture composition can be used for removing an ion, such as a sodium ion, from an ion-containing sample ingested by a subject. In embodiments, the method can comprise administering orally the ion-capture composition to a subject, before, concurrently with, or after a subject consumes an ion-containing sample.

As used herein, “sample” refers to a material comprising one or more ions. Non-limiting examples of samples include food products, drink products, and body fluids. “Food product” can refer to any substance, preparation, composition or object that is suitable for consumption, nutrition, oral hygiene or pleasure, and which is intended to be introduced into the human or animal oral cavity, to remain there for a certain period of time and then to either be swallowed or to be removed from the oral cavity again. These products include all substances or products intended to be ingested by humans or animals in a processed or a semi-processed state. As used herein, the term “drink product” can refer to a composition in the form of an ingestible substance which, at the temperature it is properly stored and ingested, is in a free-flowing liquid form, and which will not be chewed prior to swallowing. “Body fluid” can refer to any fluid obtained or obtainable from a subject. Non-limiting examples of body fluid include urine, saliva, tears, sweat, body secretions, body excretion, or any fluid that is found within, originates from or is obtained from a subject.

In embodiments, the ion-capture composition can be used to for removing ion content from a sample to reach about 1% of the original ion content, about 10% of the original ion content, about 20% of the original ion content, about 30% of the original ion content, about 40% of the original ion content, about 50% of the original ion content, about 60% of the original ion content, about 70% of the original ion content, about 80% of the original ion content, about 90% of the original ion content, about 100% of the original ion content, or less than 100% of the original ion content. In embodiments, for example, the ion removing compound can be mixed in a solution containing a known concentration of a specific ion, and after removing the compound from the solution, an ion specific meter can be used to determine the remaining ion concentration. For example, the ion specific meter can be a sodium specific ion meter, such as the Horiba LAQUA twin NA-11 compact sodium ion meter.

For example, the ion content of the sample can be reduced by about 5%, by about 10%, by about 20%, by about 30%, by about 40%, by about 50%, by about 60%, by about 70%, by about 80%, by about 90%, by about 100%, or 100%.

In embodiments, the ion content of the sample with reduced ion content is about 1% of the original ion content, about 10% of the original ion content, about 20% of the original ion content, about 30% of the original ion content, about 40% of the original ion content, about 50% of the original ion content, about 60% of the original ion content, about 70% of the original ion content, about 80% of the original ion content, about 90% of the original ion content, about 100% of the original ion content, or less than 100% of the original ion content.

Embodiments further comprise a method for selectively capturing at least one ion from the digestive system of a subject. As used herein, the digestive system can refer to the cells, tissues and organs involved in the digestion of foods, such as the stomach, duodenum, intestine and pancreas. In embodiments, the method of selectively capturing at least one ion from the digestive system of a subject comprises a monovalent ion. In embodiments, the method of selectively capturing at least one ion from the digestive system of a subject comprises a sodium ion. In some embodiments, the method of selectively capturing at least one ion comprises a sodium ion, a lithium ion, a rubidium ion, and a cesium ion.

In embodiments, methods can comprise administering to a subject a therapeutically effective amount of an ion-capture composition described herein. A “therapeutically effective amount” can refer to that amount of the therapeutic agent sufficient for a desired result. The desired result can be, for example, removing an ion from an ion-containing sample in vitro or in vivo. The desired result can also be, for example, amelioration of symptoms, e.g., treatment, healing, prevention or amelioration of the relevant medical condition, or an increase in rate of treatment, healing, prevention or amelioration of such conditions. When applied to a single active ingredient administered alone, a therapeutically effective amount can refer to that ingredient alone. When applied to a combination of ingredients administered together, a therapeutically effective amount can refer to combined amounts of the active ingredients that result in the therapeutic effect, whether administered in combination, serially or simultaneously.

In embodiments, a therapeutically effective amount can comprise a dose of about 0.005 mg/kg to about 1000 mg/kg. In some embodiments, a therapeutically effective amount can comprise a dose of about 0.005 mg/kg to about 10 mg/kg. In some embodiments, a therapeutically effective amount can comprise a dose of about 0.25 mg/kg to about 2 mg/kg. In some embodiments, the therapeutically effective amount is at least about 0.001 mg/kg at least about 0.0025 mg/kg, at least about 0.005 mg/kg, at least about 0.01 mg/kg, at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. However, the skilled artisan will recognize that the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

Embodiments as described herein can be administered to a subject in the form of a pharmaceutical compositions formulated for the intended route of administration. Such compositions can comprise, for example, the active ingredient(s) and a pharmaceutically acceptable carrier. Such compositions can be in a form adapted to oral, subcutaneous, parenteral (intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions can appear in conventional forms, for example, solutions and suspensions for injection, capsules and tablets, preferably in the form of enteric formulations, e.g. as disclosed in U.S. Pat. No. 5,350,741, for oral administration.

The pharmaceutical carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier can vary widely, such as from about 1 mg to about 1 g.

When a liquid carrier is used, the preparation can be in the form of a syrup, emulsion, soft gelatin capsule or sterile injectable liquid such as an aqueous or non-aqueous liquid suspension or solution.

The composition can also be in a form suited for local or systemic injection or infusion and can, as such, be formulated with sterile water or an isotonic saline or glucose solution. The compositions can be in a form adapted for peripheral administration only, with the exception of centrally administrable forms. The compositions can be in a form adapted for central administration.

The compositions can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The composition can contain pharmaceutically acceptable auxiliary substances as required to approximate physiological conditions, such as buffering agents, tonicity adjusting agents and the like, for example sodium acetate, sodium lactate, sodium chloride, potassium chloride, and calcium chloride.

In embodiments, the ion-capture compound of the invention can be in a pharmaceutical composition comprising a pharmaceutically acceptable carrier. A pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Multi-dose formulations can be prepared as a solution of an ion-capture compound in sterile, isotonic saline, stored in capped vials, and if necessary a preservative is added (e.g. benzoates). Fixed dose formulations can be prepared as a solution of the compound in sterile, isotonic saline, stored in glass ampoules, and if necessary filled with an inert gas. Each dose can be stored dry in ampoules or capped vials, if necessary filled with inert gas. The multi-dose formulation demands the highest degree of stability of the compound. When the stability of the compound is low fixed dose formulations can be used. For nasal administration, the preparation can contain a compound described herein dissolved or suspended in a liquid carrier, such as an aqueous carrier, for aerosol application. The carrier can contain additives such as solubilizing agents, e.g., propylene glycol, surfactants such as bile acid salts or polyoxyethylene higher alcohol ethers, absorption enhancers such as lecithin (phosphatidylcholine) or cyclodextrin, or preservatives such as parabines.

As described herein, embodiments herein can be formulated into a pharmaceutical composition to be compatible with its intended route of administration. Non-limiting examples of routes of administration include parenteral, e.g., intravenous, intradermal, subcutaneous, oral, inhalation, intranasal, transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions can be prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

The ion-capture composition can be administered orally to a subject. For example, a powder form of the ion-composition can be administered by combining the ion-capture compound in powder form with a solution, stirring until a suspension forms, and ingesting the suspension containing the ion-capture composition. Other examples include, but are not limited to, placing a tablet, capsule, or other form of the ion-capture composition in an oral cavity and ingesting the ion-capture composition before, concurrently with, or after the ingestion of an ion-containing substance or sample.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral compositions can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In embodiments, administering can comprise the placement of a pharmaceutical composition, such as a composition comprising a botanical extract, into a subject by a method or route which results in at least partial localization of the composition at a desired site such that desired effect is produced.

For example, the pharmaceutical composition can be administered by bolus injection or by infusion. A bolus injection can refer to a route of administration in which a syrine is connected to the IV access device and the medication is injected directly into the subject. The term “infusion” can refer to an intravascular injection.

In embodiments, the ion-capture composition can comprise administering to a subject the ion-capture composition in a single dose or in multiple doses. For example, embodiments as described herein can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration of the botanical extract in a continuous manner, such as to maintain the therapeutic effect (activity) over a prolonged period of time.

In embodiments, the term “subject” or “patient” can refer to any organism to which aspects of the invention can be administered, e.g., for experimental, diagnostic, prophylactic, and/or therapeutic purposes. Typical subjects to which compounds described herein can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject. The terms “subject”, “individual”, and “patient” can be used interchangeably.

In embodiments, the capture of an ion by the ion-capture composition can be through chemical means, physical means, or both. For example, a physical means can refer to physiosorption. For example, a chemical means can refer to chemisorption. In one embodiment, the physical means encompass ions experiencing an electronegative attraction towards the pores of the ion-capturing composition; once the composition has pulled the ions in, the small size of the pores act as a physical barrier, making it difficult for the ions to escape the electronegative pull of the pores.

In embodiments, at least one ion is captured in the digestive tract. As used herein, the term “digestive tract” can refer to the system of organs within multicellular animals that takes in food, digests it to extract energy and nutrients, and expels the remaining waste. Functions of the GI tract include digestion and excretion. The upper GI tract comprises the mouth, pharynx, esophagus, and stomach and the lower GI tract comprises the organs beneath the stomach, e.g., the intestines, colon, and anus. The small intestine comprises the duodenum, jejunum, and ileum and the large intestine comprises the cecum, colon (ascending colon, transverse colon, descending colon and sigmoid flexure), and rectum. In embodiments, for example, the subject can ingest the ion-capturing composition, at least one ion is captured in the digestive tract of the subject, and then the ion-capturing composition can be recovered after excretion from the body and subsequently treated with an acidic solution (>pH 5) to remove any ions captured by the compound while inside the body. The removed ions could then be measured by an ion specific meter.

In embodiments, the ion-capture composition can be administered to a subject about 1 milligram to about 100 grams per day. For example, the subject can be administered about 1 milligram, about 10 milligrams, about 100 milligrams, about 1 gram, about 2 grams, about 5 grams, about 10 grams, about 20 grams, about 25 grams, about 30 grams, about 35 grams, about 40 grams, about 45 grams, about 50 grams, about 75 grams, or about 100 grams per day. For example, hydrogen zirconium phosphate removes sodium ions from solution with a ratio of 45.7 mg of sodium/1 g of hydrogen zirconium phosphate. Currently the American Heart Association recommends that Americans reduce their sodium intake by around 1000 mg per day which would result in a dosage of about 22 g per day (https://www.heart.org/-/media/data-import/downloadables/8/2/0/pe-abh-why-should-i-limit-sodium-ucm_300625.pdf). The amount of the ion-capture composition to be administered to a subject can be administered in a single dose (i.e., all at once) or in multiple doses (i.e., multiple doses throughout the day). “Sodium intake” can refer to the total amount of salt, or such as sodium, e.g., sodium chloride, an organism receives from external sources such as nutrition (food and liquids), or liquid infusion. Chemically defined, the term “salt” can refer to an ionic molecule, where two or more ions of opposing charges are bound together through electrostatic interactions. For example, the term can refer to sodium chloride (NaCl), due to the widespread usage of NaCl in chemistry as well as in preparing, seasoning, and/or preserving foods. For example, “table salt” can refer to a specific food ingredient which is used for preparing, seasoning, and/or preserving foods.

Aspects of the invention can further be used to treat or prevent a disease or condition. For example, a “disease” or “disorder” can refer to an abnormal condition of an organism that impairs bodily functions, associated with specific symptoms and signs. Examples of diseases and disorders can include, without limitation, heart disease, hypertension, congestive heart failure, chronic kidney disease, kidney failure and edema.

As used herein, the term “heart disease” can refer to a cardiac condition or a condition of the heart prior to the onset of heart failure.

The term “hypertension” can refer to a disorder in which the blood pressure in the pulmonary arteries is abnormally high.

“Congestive heart failure” can refer to an impaired cardiac function that renders the heart unable to maintain the normal blood output at rest or with exercise, or to maintain a normal cardiac output in the setting of normal cardiac filling pressure.

As used herein, the term “chronic kidney disease” can refer to a disease or condition associated with impaired kidney function which can cause kidney damage over time.

The term “kidney failure” can refer to a condition in which the quantitative and qualitative homeostasis of body fluids cannot be maintained due to insufficient blood excretion of nitrogen metabolites, water, and electrolytes due to renal failure, nephron loss, and urinary obstruction.

“Edema” can refer to swelling in any tissue or organ due to an increase in interstitial fluid caused by vascular leakage.

In embodiments, the term “treating” or “to treat” can refer to clinical intervention in an attempt to alter the natural course of the individual or subject being treated. For example, “treating a disease” can comprise curing a disease, preventing a disease, reducing the incidence of a disease, or ameliorating symptoms of a disease.

Kits

Aspects of the invention are also directed towards kits for removing one or more ions from an ion-containing sample. The term “kit” can refer to a set of articles that facilitates the process, method, assay, analysis or manipulation of a sample. The kit can include instructions for using the kit (eg, instructions for the method of the present invention), materials, solutions, components, reagents, chemicals, or enzymes required for the method, and other optional components. In embodiments, the invention provides kits for removing one or more ions from an ion-containing sample, thus reducing the ion content of the ion-containing sample. In some embodiments, the kits comprise of a flavor additive. Non-limiting examples of a flavor additive include potassium salts. In some embodiments, the kits comprise a containment apparatus that protects the composition from unwanted gas or liquid contact.

In embodiments, the ion-capture composition can be provided in a kit. For example, the kit can include (a) a container that contains a ion-capture composition that includes the ion-capture compound, and optionally (b) informational material. The informational material can be descriptive, instructional, marketing or other material that relates to the methods described herein and/or the use of the agents for therapeutic benefit. In embodiments, the kit can also include one or more additional agents, such as a second agent for treating a disease or condition. For example, the kit includes a first container that contains the ion-capture composition, and a second container that includes the second agent.

The informational material of the kits is not limited in its form. In embodiments, the informational material can include information about production of the ion-capture compound, molecular weight of the ion-capture compound, concentration, date of expiration, batch or production site information, and so forth. In one embodiment, the informational material relates to methods of administering the botanical extract, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

In addition to the ion-capture compound and/or composition, the composition in the kit can include other ingredients, such as a solvent or buffer, a stabilizer, or a preservative. The ion-capture composition or components thereof can be provided in any form, e.g., liquid, dried or lyophilized form, and can be substantially pure and/or sterile. When the ion-capture composition is provided in a liquid solution, the liquid solution can be an aqueous solution or an alcohol solution. When the ion-capture composition or components thereof are provided as a dried form, reconstitution generally is by the addition of a suitable solvent. The solvent, e.g., sterile water or buffer, can optionally be provided in the kit.

The kit can include one or more containers for the composition or compositions containing the agents. In embodiments, the kit contains separate containers, dividers or compartments for the composition and informational material. For example, the composition can be contained in a bottle, vial, or syringe, and the informational material can be contained in a plastic sleeve or packet. In embodiments, the separate elements of the kit are contained within a single, undivided container. For example, the composition can be contained in a bottle, vial or syringe that has attached thereto the informational material in the form of a label. In embodiments, the kit can include a plurality (e.g., a pack) of individual containers, each containing one or more unit dosage forms (e.g., a dosage form described herein) of the agents. The containers can include a combination unit dosage, e.g., a unit that includes the botanical extract and the second agent, e.g., in a desired ratio. For example, the kit can include a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight. The kit optionally can include a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Synthesis of Zirconium Phosphate

Add 40 g zirconyl chloride octahydrate, 31.30 g oxalic acid, and 19.361 g monosodium phosphate to 500 mL distilled water in a large round-bottom flask. Stir and continue adding distilled water until all three powders have dissolved. Add ammonium hydroxide in small portions until the solution has a pH of 3. Reflux with an air condenser for six days; the solution will be milky white in color. Filter the precipitate using a fritted funnel*. Stir filtered precipitate with 210 mL distilled water and 90 mL 36.5% hydrochloric acid solution for 24 hours. Filter the precipitate using a fritted funnel. Wash the solution with distilled water, ensuring that no acid remains. Remove as much liquid as possible from the precipitate using a fritted funnel. Heat precipitate at 680 degrees Celsius for one hour in an oven to heat treat. Allow to cool and weigh precipitate. Total weight of precipitate obtained was 19.874 g.

*Filtering the precipitate in a fritted funnel was extremely difficult due to its small particle size. Centrifugation will likely be much easier and will be pursued when this synthesis is repeated.

Testing:

The goal of this testing was to test the sodium selectivity of the synthesized zirconium phosphate as well as to test the effect the concentration of zirconium phosphate used affected the sodium removal time.

Prepare three test solutions of zirconium phosphate.

1. 1.011 g zirconium phosphate and 6.4 mL distilled water
a. This allows for a 1 g zirconium phosphate: 45.7 mg sodium ratio.

2. 2.022 g zirconium phosphate and 6.4 mL distilled water
a. This allows for a 2 g zirconium phosphate: 45.7 mg sodium ratio.

3. 4.044 g zirconium phosphate and 6.4 mL distilled water
a. This allows for a 4 g zirconium phosphate: 45.7 mg sodium ratio.

(45.7 mg is significant because the paper that describes the sodium selective nature of zirconium phosphate states that 1 gram of zirconium phosphate can adsorb 45.7 mg of sodium. Varying the ratio of zirconium phosphate to sodium allows us to see the impact a higher concentration of zirconium phosphate has on the rate of sodium removal.)

Prepare a sodium chloride solution with a sodium concentration of 7400 ppm. Boil down Swanson's chicken broth until its sodium concentration reaches 7400 ppm (The normal sodium concentration of Swanson's chicken broth is 3700 ppm. Doubling the sodium concentration to 7400 ppm allows the water in the zirconium phosphate solution to dilute the entire test solution to 3700 ppm when they are mixed).

Obtain and label 90 1.5-mL microcentrifuge tubes by number:
Tubes 1-15, 46-60: Add 218 microliters of zirconium phosphate solution 1
Tubes 16-30, 61-75: Add 209 microliters of zirconium phosphate solution 2
Tubes 31-45, 76-90: Add 204.5 microliters of zirconium phosphate solution 3
Batch 1: Add 200 microliters of sodium chloride solution to tubes 1, 16, and 31, and 200 microliters of concentrated chicken broth solution to tubes 46, 61, and 76. Cap tubes and place in agitating incubator set at 80 degrees Celsius.
Batch 2: Add 200 microliters of sodium chloride solution to tubes 2, 17, and 32, and 200 microliters of concentrated chicken broth solution to tubes 47, 62, and 77. Cap tubes and place in agitating incubator set at 80 degrees Celsius.
Batch 3: Add 200 microliters of sodium chloride solution to tubes 3, 18, and 33, and 200 microliters of concentrated chicken broth solution to tubes 48, 63, and 78. Cap tubes and place in agitating incubator set at 80 degrees Celsius.
Batch 4: Add 200 microliters of sodium chloride solution to tubes 4, 19, and 34, and 200 microliters of concentrated chicken broth solution to tubes 49, 64, and 79. Cap tubes and place in agitating incubator set at 80 degrees Celsius.
Batch 5: Add 200 microliters of sodium chloride solution to tubes 5, 20, and 35, and 200 microliters of concentrated chicken broth solution to tubes 50, 65, and 80. Cap tubes and place in agitating incubator set at 80 degrees Celsius.
Batch 6: Add 200 microliters of sodium chloride solution to tubes 6, 21, and 36, and 200 microliters of concentrated chicken broth solution to tubes 51, 66, and 81. Cap tubes and place in agitating incubator set at 80 degrees Celsius.
Batch 7: Add 200 microliters of sodium chloride solution to tubes 7, 22, and 37, and 200 microliters of concentrated chicken broth solution to tubes 52, 67, and 82. Cap tubes and place in agitating incubator set at 80 degrees Celsius.
Batch 8: Add 200 microliters of sodium chloride solution to tubes 8, 23, and 38, and 200 microliters of concentrated chicken broth solution to tubes 53, 68, and 83. Cap tubes and place in agitating incubator set at 80 degrees Celsius.
Batch 9: Add 200 microliters of sodium chloride solution to tubes 9, 24, and 39, and 200 microliters of concentrated chicken broth solution to tubes 54, 69, and 84. Cap tubes and place in agitating incubator set at 80 degrees Celsius.
Batch 10: Add 200 microliters of sodium chloride solution to tubes 10, 25, and 40, and 200 microliters of concentrated chicken broth solution to tubes 55, 70, and 85. Cap tubes and place in agitating incubator set at 80 degrees Celsius.
Batch 11: Add 200 microliters of sodium chloride solution to tubes 11, 26, and 41, and 200 microliters of concentrated chicken broth solution to tubes 56, 71, and 86. Cap tubes and place in agitating incubator set at 80 degrees Celsius.
Batch 12: Add 200 microliters of sodium chloride solution to tubes 12, 27, and 42, and 200 microliters of concentrated chicken broth solution to tubes 57, 72, and 87. Cap tubes and place in agitating incubator set at 80 degrees Celsius.
Batch 13: Add 200 microliters of sodium chloride solution to tubes 13, 28, and 43, and 200 microliters of concentrated chicken broth solution to tubes 58, 73, and 88. Cap tubes and place in agitating incubator set at 80 degrees Celsius.
Batch 14: Add 200 microliters of sodium chloride solution to tubes 14, 29, and 44, and 200 microliters of concentrated chicken broth solution to tubes 59, 74, and 89. Cap tubes and place in agitating incubator set at 80 degrees Celsius.
Batch 15: Add 200 microliters of sodium chloride solution to tubes 15, 30, and 45, and 200 microliters of concentrated chicken broth solution to tubes 60, 75, and 90. Cap tubes and place in agitating incubator set at 80 degrees Celsius.

Remove tubes from incubator after they have been in the incubator for the following lengths of time:
Batch 1: 15 minutes
Batch 2: 30 minutes
Batch 3: 45 minutes
Batch 4: 60 minutes
Batch 5: 75 minutes
Batch 6: 90 minutes
Batch 7: 105 minutes
Batch 8: 120 minutes
Batch 9: 135 minutes
Batch 10: 150 minutes
Batch 11: 165 minutes
Batch 12: 180 minutes
Batch 13: 195 minutes
Batch 14: 210 minutes
Batch 15: 225 minutes

Once each batch has been removed from the incubator, centrifuge each tube in a microcentrifuge at top speed for 20 seconds. Remove from microcentrifuge and pipette off the liquid from each tube and place into new microcentrifuge tube. Test sodium concentration with LAQUA Sodium Ion Meter. (Note: Water began evaporating from tubes after they had been in the incubator for a while. As such, there was not enough liquid in the tubes after 105 minutes for the sodium meter to register a concentration when tested.)

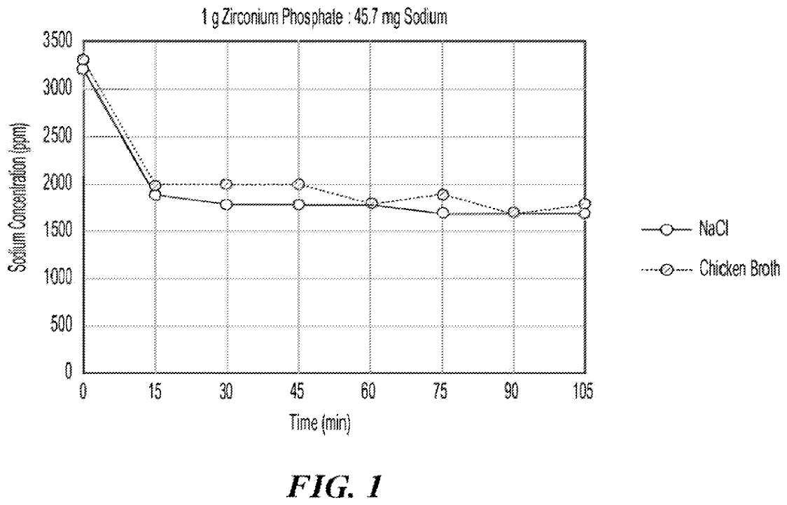

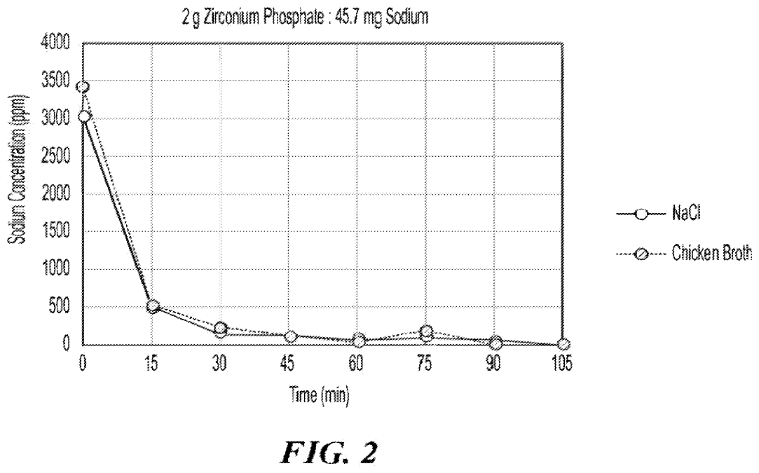

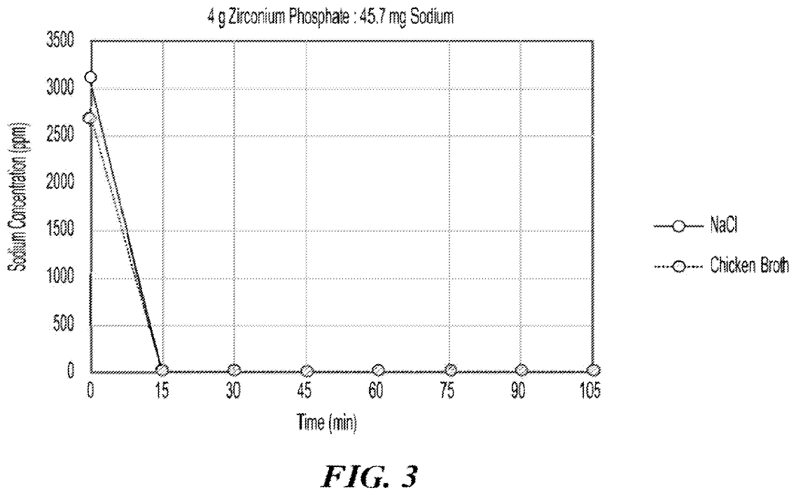

The results of these tests can be seen in FIGS. 1-4. In FIGS. 1-4, the sodium content over time looks almost identical for both the NaCl solution and the Chicken Broth. This suggests that the compound synthesized was indeed sodium selective, as chicken broth contains many non-sodium ions that may have been competing with sodium ions for zirconium phosphate adsorption. The data from FIG. 5 was not very useful, since all the sodium was removed in the first 15 minutes. As such, a similar test was run, this time only using 4 g Zirconium Phosphate: 45.7 mg Sodium and only Chicken Broth.

### Example 2—Test 2

Prepare a test solution with 0.5184 g zirconium phosphate and 3.2 mL distilled water. Number microcentrifuge tubes 1-15. Add 218 microliters of test solution to each tube. Add 200 microliters of concentrated chicken broth to each tube. Add to agitating incubator set at 80 degrees Celsius.

Remove tubes after the following amounts of time:
Tube 1: 1 minute
Tube 2: 2 minutes
Tube 3: 3 minutes
Tube 4: 4 minutes
Tube 5: 5 minutes
Tube 6: 6 minutes
Tube 7: 7 minutes
Tube 8: 8 minutes
Tube 9: 9 minutes
Tube 10: 10 minutes
Tube 11: 11 minutes
Tube 12: 12 minutes
Tube 13: 13 minutes
Tube 14: 14 minutes
Tube 15: 15 minutes

Once each tube has been removed from the incubator, centrifuge each tube at top speed for 20 seconds. Remove from microcentrifuge and pipette off the liquid from each tube and place into new microcentrifuge tube. Test sodium concentration with LAQUA Sodium Ion Meter.

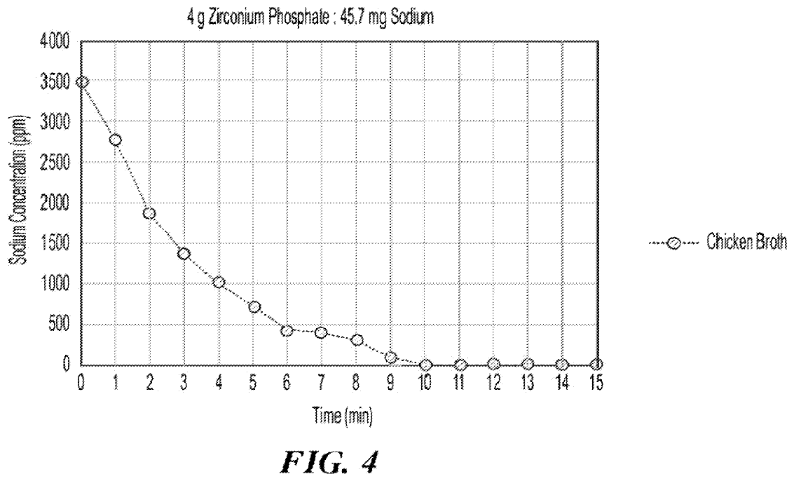

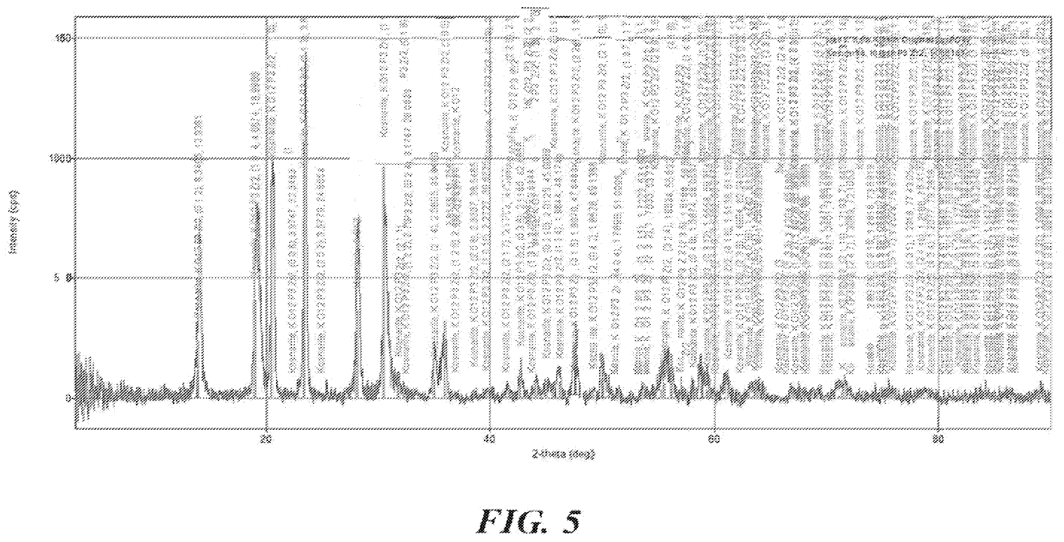

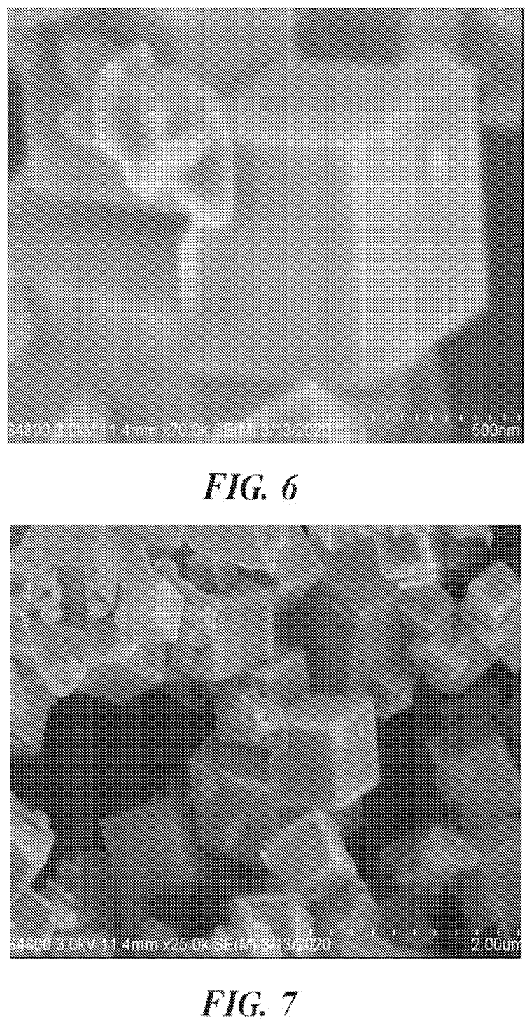

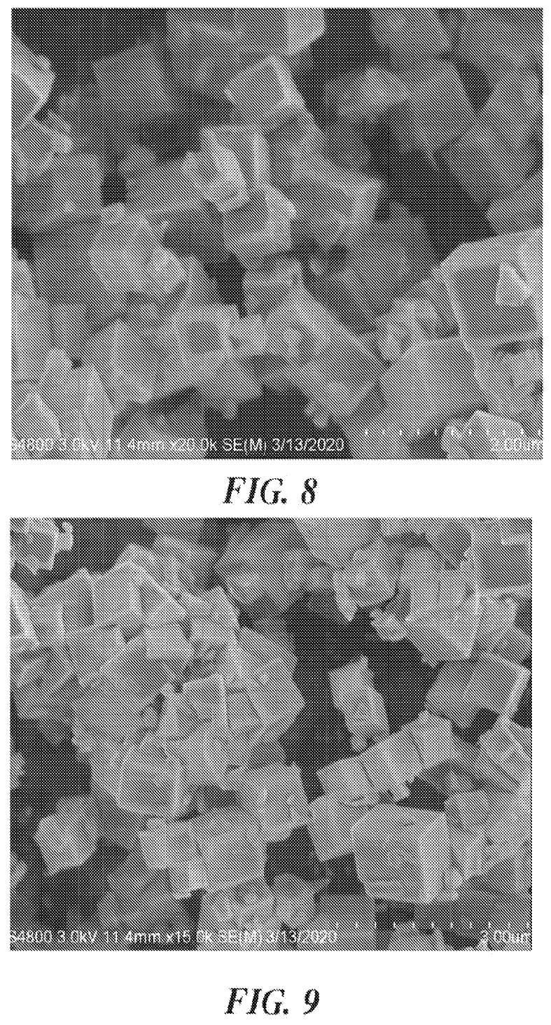

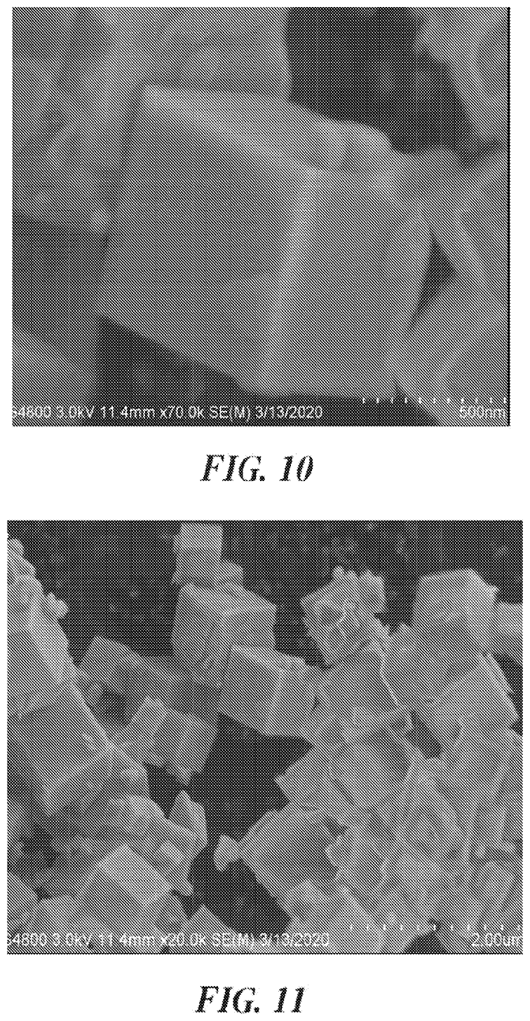

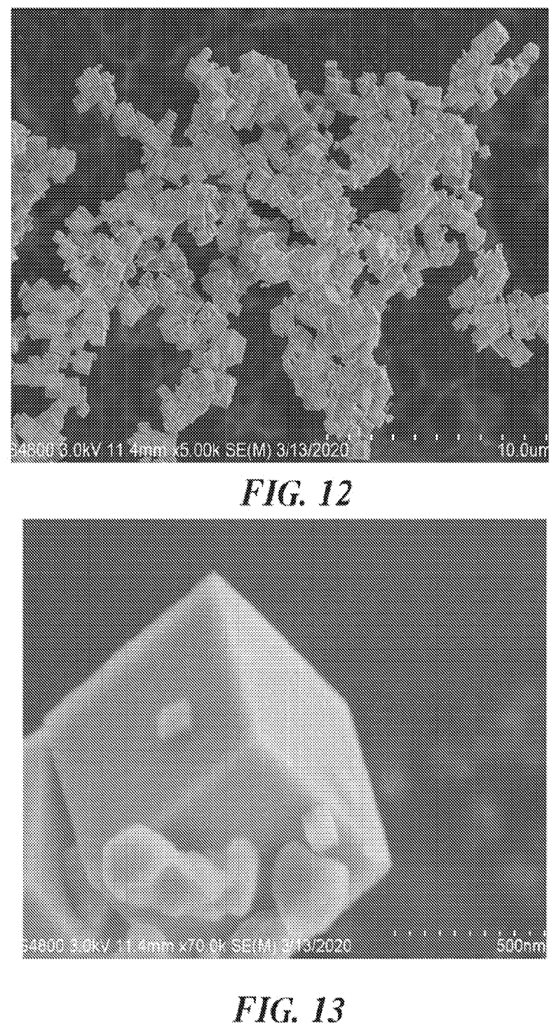

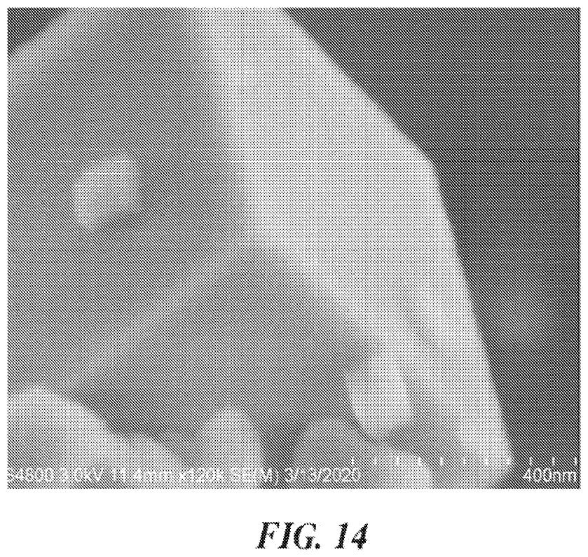

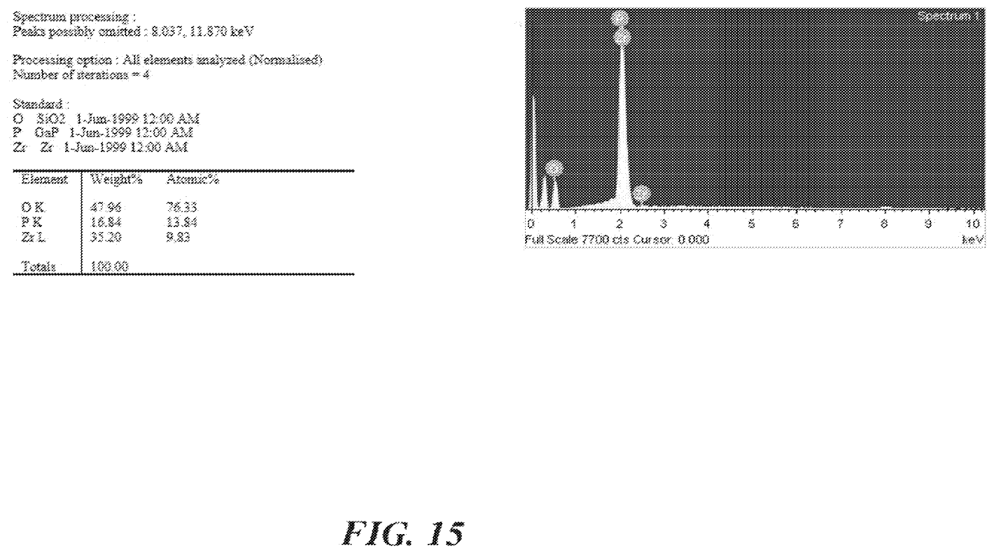

The result of this test can be seen in FIG. 4, below.

This test showed that all of the sodium was removed from the chicken broth in only 10 minutes and that sodium removal begins rapidly and proceeds more slowly as time goes on.

### REFERENCES CITED IN THIS EXAMPLE

[1] Nakajima, Yasushi, and Isao Yoshida. “Sodium Selective Ion-Exchange Properties of Zirconium Phosphate, HZr₂(PO₄)₃, and Its Application for the Removal of Sodium Ions.” Analytical Sciences, vol. 12, no. 6, Dec. 1996, pp. 935-940., doi:10.2116/analsci.12.935.
[2] Keyser, D. J., Guillem, A. F. (2014). U.S. Pat. No. 8,877,255 B2 Retrieved from https://patentimages.storage.googleapis.com/a9/82/68/951427cca7a293/U.S. Pat. No. 8,877,255.pdf
[3] “Health Risks and Disease Related to Salt and Sodium.” The Nutrition Source, Harvard School of Public Health, 6 Jul. 2016, www.hsph.harvard.edu/nutritionsource/salt-and-sodium/sodium-health-risks-and-disease/.
[4] Semplicini, Andrea. “Faculty of 1000 Evaluation for Worldwide Trends in Blood Pressure from 1975 to 2015: a Pooled Analysis of 1479 Population-Based Measurement Studies with 19.1 Million Participants.” F1000—Post-Publication Peer Review of the Biomedical Literature, 2016, doi:10.3410/11726994163.793525652.
[5] “Heart Disease Facts & Statistics.” Centers for Disease Control and Prevention, Centers for Disease Control and Prevention, 28 Nov. 2017, www.cdc.gov/heartdisease/facts.htm.

### Example 3—Ion-Capture Composition for Ingestion

The substance responsible for the sodium-selective capture of ions can be any substance that at least somewhat specifically captures sodium ions for removal. For example, and without wishing to be bound by theory, the ion-capture does not have to only remove sodium ions, but it should have at least some specificity for sodium ions over other ions. The substance can do so through ion-exchange, trapping ions within a crystal structure of the substance, adsorption, chelation, or through any other similar chemical or physical means. One example of such a substance is hydrogen zirconium phosphate, HZr₂(PO₄)₃ as described by Y. Nakajima and I. Yoshida in their paper, “Sodium Selective Ion-Exchange Properties of Zirconium Phosphate, HZR₂(PO₄)₃, and Its Application for the Removal of Sodium Ions” (Analytical Sciences 12 (6), 935-940, 1996). This hydrogen zirconium phosphate can be ingested by a subject to remove ions from an ingested sample.

Zirconium Phosphate Powder (and possibly a food safe buffer).
1. Add zirconium phosphate powder to water (or other beverage)
2. Stir until a suspension forms
3. Drink (before or after a meal)

Zirconium Phosphate Chewable Tablet (containing zirconium phosphate and possible binders, flavorings, colorants, and food safe buffers)
1. Place zirconium phosphate tablet(s) in mouth (before or after a meal)
2. Chew until tablet has been fully consumed

Zirconium Phosphate Ready-Made Suspension (containing zirconium phosphate and possible water, chemical to maintain suspension, flavorings, colorants, and food safe buffers)
1. Measure out a certain amount of suspension (or use pre-measured amount)
2. Drink (before or after a meal)

Zirconium Phosphate Swallowable Pill (containing zirconium phosphate and possible binders, colorants, and food safe buffers)
1. Place zirconium phosphate pill(s) in mouth (before or after a meal)
2. Swallow pill

The amount of zirconium phosphate consumed depends on the amount of sodium removal desired. 1 gram of zirconium phosphate will remove around 45.7 mg of sodium from the digestive system. After consumption, the zirconium phosphate is passed through the body and is excreted in the stool.

### Example 4—Ion-Capture Composition for Ingestion as a Diuretic Replacement

One current treatment for patients with hypertension is the use of diuretics, which remove excess sodium and water from the body through the kidneys. While treatment with diuretics is very common, there are some harmful side effects that are associated with diuretic use. For example, many diuretics are not sodium-specific, meaning they remove an assortment of other ions from the body, as well as sodium. When too many other ions are removed, this can sometimes be harmful, leading to conditions such as hypokalemia (low serum potassium levels) [1], hypocalcemia (low serum calcium levels) [3] hypomagnesemia (low serum magnesium levels) [3], as well as other harmful ion deficiency conditions. Because the ion-capture composition for ingestion described herein can be sodium-specific, other essential ions are not lost through treatment with the composition. Thus, without wishing to be bound by theory, these harmful conditions associated with diuretic use will not occur.

Also, it has been shown that chronic diuretic use can negatively impact glucose homeostasis. Hypertension and diabetes are comorbidities and, as such, treatment of hypertension with diuretics could prove severely harmful to patients with diabetes [4].

Further, while diuretics can be used to treat hypertension to prevent acute kidney injury and progression to chronic kidney disease, research has shown that diuretics are actually associated with heightened risk for acute kidney injury [5]. In addition, high dosages or prolonged usage of certain diuretics can also harm the kidneys [6].

Still further, without wishing to be bound by theory, because of its low solubility in water, the ion-capture composition for ingestion described herein is not bioactive. As a result, there is a low risk of interactions with other medications. Contrastingly, as diuretics are bioactive pharmaceuticals, there is a litany of other medications that may interact with diuretics including, but not limited to, “cyclosporine (Restasis), antidepressants such as fluoxetine (Prozac) and venlafaxine (Effexor XR), lithium, digoxin (Digox),” [7] “antihypertensive agents, potassium, magnesium or acid salts, probenecid, quinidine, anticoagulants, lithium, cardiac glycosides . . . laxatives, oral antidiabetic agents, non-steroidal anti-inflammatory drugs, adenylate cyclase activators, mineralocorticoids, hypolipidemic agents, neuromuscular blockers, chloral hydrate, carbenoxolone, drugs likely to produce the syndrome of inappropriate secretion of antidiuretic hormone, and some antibiotics” [8].

As a result, without wishing to be bound by theory, the ion-capture composition for ingestion described herein can be used as a supplement, complement, and/or replacement to diuretics in the treatment of a variety of diseases and conditions, including but not limited to hypertension, heart failure and kidney disease.

### REFERENCES CITED IN THIS EXAMPLE

[1] “Diuretics.” Diuretics, Mayo Clinic, 19 Oct. 2019, https://www.mayoclinic.org/diseases-conditions/high-blood-pressure/in-depth/diuretics/art-20048129.
[2] “Diuretics—Adverse Effects.” Open Anesthesia, Open Anesthesia, https://www.openanesthesia.org/aba_diuretics_-adverse_effects/#:˜:text=Thiazide %20diuretics %20can %20cause %20hypercalcemia,cause % 20ototoxicity %20and %20hearing %20loss
[3] Efstratiadis, G., M. Sarigianni, and I. Gougourelas. “Hypomagnesemia and Cardiovascular system.” Hippokratia, 10.4 (2006): 147.
[4] Buscemi, Silvio, et al. “Impact of Chronic Diuretic Treatment on Glucose Homeostasis.” Diabetology & Metabolic Syndrome, 5.1 (2013): 1-8.
[5] Ejaz, A. A., & Mohandas, R. “Are Diuretics Harmful in the Management of Acute Kidney Injury?” Current Opinion in Nephrology and Hypertension, 23.2 (2014): 155-160.
[6] Kuznar, W. “High Furosemide Dosage May Harm Kidneys.” Cardiovascular Disease (CVD), Renal & Urology News, 20 Nov. 2013, https://www.renalandurologynews.com/home/news/nephrology/cardiovascular-disease-cvd/high-furosemide-dosage-may-harm-kidneys/[7]
[7] Carter, A. “What to Know About Diuretics.” Diuretics, Healthine, 27 Feb. 2019, https://www.healthline.com/health/diuretics
[8] Leary W P, Reyes A J. “Drug Interactions with Diuretics.” South African Medical Journal, 65.12 (1984): 455-461.

### Example 5

Aspects of the invention comprise ion-capture compositions that can remove salt from food thus reducing sodium intake into the body. Embodiments comprise a process that is an inorganic reaction yielding zirconium phosphate as the key component.

An embodiment of the process is as follows: add 40 g zirconyl chloride octahydrate, 31.30 g oxalic acid, and 19.36 g monosodium phosphate to 500 mL distilled water in a 2-liter round-bottom flask. Stir and continue adding distilled water until all three powders have (at least partially) dissolved. Add ammonium hydroxide in small portions until the solution reaches a pH of 3. Leave reaction mixture to reflux with a condenser for six days; the solution will become milky white in color. Filter the precipitate. Stir filtered precipitate with 210 mL distilled water and 90 mL 36.5% hydrochloric acid solution for 24 hours. Filter the precipitate. Wash the solution with distilled water, ensuring that no acid remains. Remove as much liquid as possible from the precipitate. Heat precipitate at 680 degrees Celsius for one hour in an oven to heat treat. Allow to cool and weigh precipitate.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An ion-capture composition, the composition comprising an ion-capture compound that traps at least one ion from a sample ingested by a subject.

2. The ion-capture composition of claim 1, wherein the compound comprises the formula M′M″ₓA₂B₃O₁₂ in NZP form.

3. The composition of claim 1, wherein the composition further comprises a pharmaceutically acceptable carrier.

4. The composition of claim 1, wherein the ion-capture compound comprises microscopic cubic crystals.

5. The composition of claim 1, wherein the ion-capture composition comprises a powder, a granule, a crystal, a suspension, an emulsion, a solution, or a resin.

6. The composition of claim 1, wherein the composition is provided as a powder, a tablet, a capsule, or a pre-made suspension.

7. The composition of claim 1, wherein the composition is formulated for oral administration.

8. The composition of claim 1, wherein the composition is formulated to be ingested by the subject.

9. The composition of claim 1, wherein the at least one ion comprises a monovalent ion.

10. The composition of claim 1, wherein the at least one ion comprises a sodium ion.

11. The composition of claim 1, wherein the at least one ion comprises a sodium ion, lithium ion, a rubidium ion, or a cesium ion.

12. The composition of claim 1, wherein the ion-capture comprises chemical capture, physical capture, or both.

13. The composition of claim 1, wherein the compound captures an ion through ion-exchange, trapping ions within the crystal structure of the compound, adsorption, or chelation.

14. The composition of claim 2, wherein
M′ comprises a lithium ion, sodium ion, potassium ion, hydrogen ion, hydronium ion, rubidium ion, cesium ion, magnesium ion, calcium ion, strontium ion, barium ion, or a combination thereof;
M″ comprises a sodium ion, potassium ion, hydrogen ion, hydronium ion, or a combination thereof;
A comprises tin, germanium, titanium, zirconium, hafnium, aluminum, chromium, nobium, tantalum, scandium, or combinations thereof;
B comprises phosphorous, silicon, aluminum, sulfur, or mixtures thereof; and
“x” has a value of 0 to about 3.

15. The composition of claim 14, wherein the compound comprises hydrogen zirconium phosphate, or HZr₂(PO₄)₃.

16. The composition of claim 5, wherein the powder form is combined with a solution, thereby forming a suspension.

17. A pharmaceutical composition comprising the composition of claim 1.

18. The composition of claim 16, wherein the composition comprises a buffer.

19. A method for capturing at least one ion from a sample ingested by a subject, the method comprising administering orally to the subject the composition of claim 1, wherein the composition is administered before, concurrently with, or after the subject ingests a sample comprising one or more ions.

20. A method for selectively capturing at least one ion from the digestive system of a subject, the method comprising administering orally to the subject the composition of claim 1.

21. The method of claim 19, wherein the at least one ion comprises a monovalent ion.

22. The method of claim 19, wherein the at least one ion comprises a sodium ion.

23. The method of claim 19, wherein the at least one ion comprises a sodium ion, lithium ion, a rubidium ion, or a cesium ion.

24. The method of claim 19, wherein the ion-capture comprises chemical capture, physical capture, or both.

25. The method of claim 19, wherein the compound captures an ion through ion-exchange, trapping ions within the crystal structure of the substance, adsorption, or chelation.

26. The method of claim 20, wherein the at least one ion is captured in the digestive tract.

27. The method of claim 20, wherein the subject is administered about 1 milligram to about 100 grams per day.

28. A method of treating a subject afflicted with a disease, the method comprising administering orally to a subject the composition of claim 1.

29. The method of claim 28, wherein the disease comprises heart disease, hypertension, congestive heart failure, chronic kidney disease, kidney failure or edema.
